(19) United States
(12) Patent Application Publication
(10) Pub. No.: US 2014/0014105 A1
(43) Pub. Date: Jan. 16, 2014

(54) DRY POWDER INHALER WITH LARGE CAPACITY RESERVOIR

(71) Applicants: Annaniy Berenshteyn, Ocean, NJ (US); Mikhail Gotliboym, Scotch Plains, NJ (US); Aleksandr Zuyev, Denville, NJ (US)

(72) Inventors: Annaniy Berenshteyn, Ocean, NJ (US); Mikhail Gotliboym, Scotch Plains, NJ (US); Aleksandr Zuyev, Denville, NJ (US)

(21) Appl. No.: 13/939,765

(22) Filed: Jul. 11, 2013

Related U.S. Application Data
(60) Provisional application No. 61/670,795, filed on Jul. 12, 2012.

(51) Int. Cl. A61M 15/00

(52) U.S. Cl. CPC A61M 15/0065
USPC 128/203.15

(57) ABSTRACT

A dry powder inhaler is provided herein having a powder housing defined on an exterior by a generally cylindrical wall. Within the interior volume of the wall, a central passageway is provided through which certain components of the dry powder inhaler may pass. In addition, a powder reservoir is defined within the interior volume having a generally crescent-shaped profile formed along a substantial length of the wall. The powder reservoir terminates at end points between which is defined an isolated inhalation channel. With the configuration provided herein, the powder reservoir has a substantial working length exposed at its bottom to permit removal of medication therefrom. Advantageously, with this configuration, a relatively large amount of medication may be maintained, and obtained access to, in the powder reservoir.

### FIELD OF THE INVENTION

The present invention relates to dry powder inhalers and, more particularly, to dry powder inhalers having large capacity reservoirs.

### BACKGROUND OF THE INVENTION

Dry powder inhalers are well known in the art. U.S. Pat. No. 6,240,918 discloses a well known design. U.S. Pat. No. 6,240,918 is incorporated by reference herein in its entirety.

In spite of known designs, situations arise where large number of doses and/or the ability to provide a meaningful number of large volume doses are needed. There is a need for dry powder inhalers which have large capacity reservoirs to accommodate sufficient medication to not only provide a relatively high number of doses, but also to provide a meaningful number of large volume doses, as needed.

### SUMMARY OF THE INVENTION

A dry powder inhaler is provided herein having a powder housing defined on an exterior by a generally cylindrical wall. Within the interior volume of the wall, a central passageway is provided through which certain components of the dry powder inhaler may pass. In addition, a powder reservoir is defined within the interior volume having a generally crescent-shaped profile formed along a substantial length of the wall. The powder reservoir terminates at end points between which is defined an isolated inhalation channel. With the configuration provided herein, the powder reservoir has a substantial working length exposed at its bottom to permit removal of medication therefrom. Advantageously, with this configuration, a relatively large amount of medication may be maintained, and obtained access to, in the powder reservoir.

These and other features of the invention will be better understood through a study of the following detailed description and accompanying drawings.

### BRIEF DESCRIPTION OF THE DRAWINGS

FIGS. 1-25 depict a dry powder inhaler, and components thereof, formed in accordance with the subject invention.

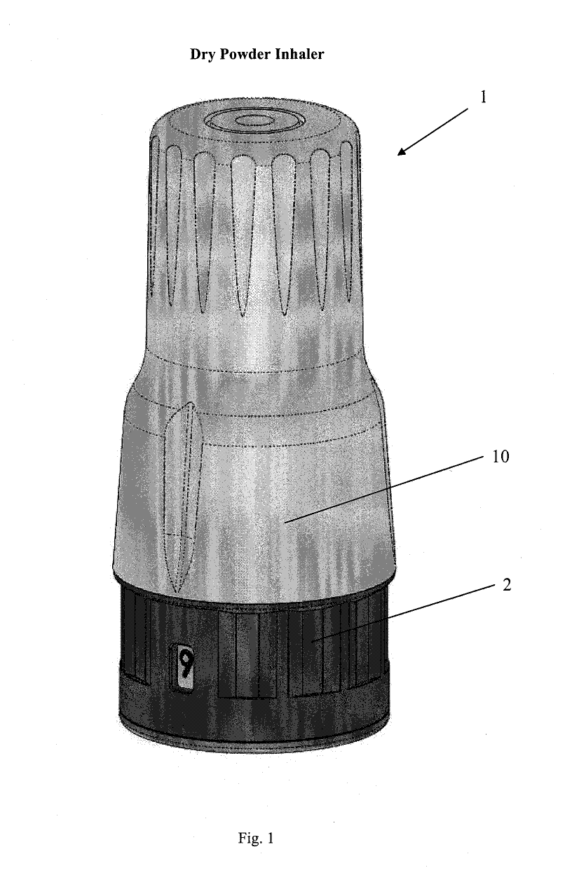

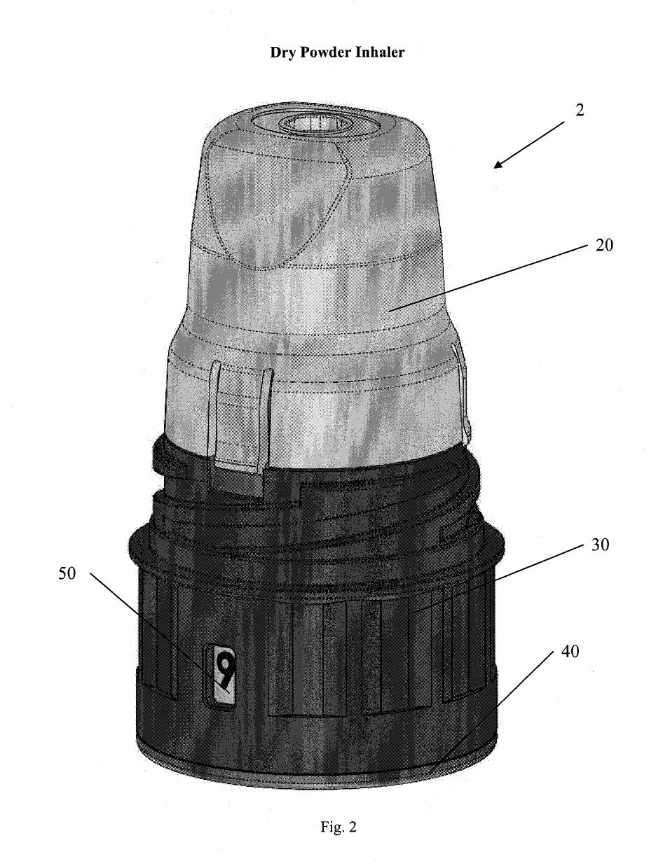

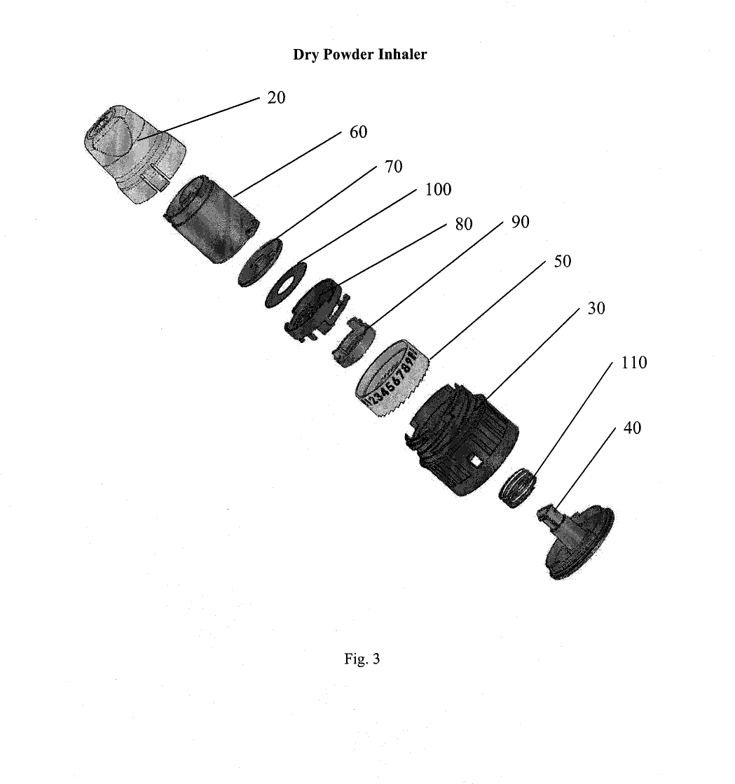

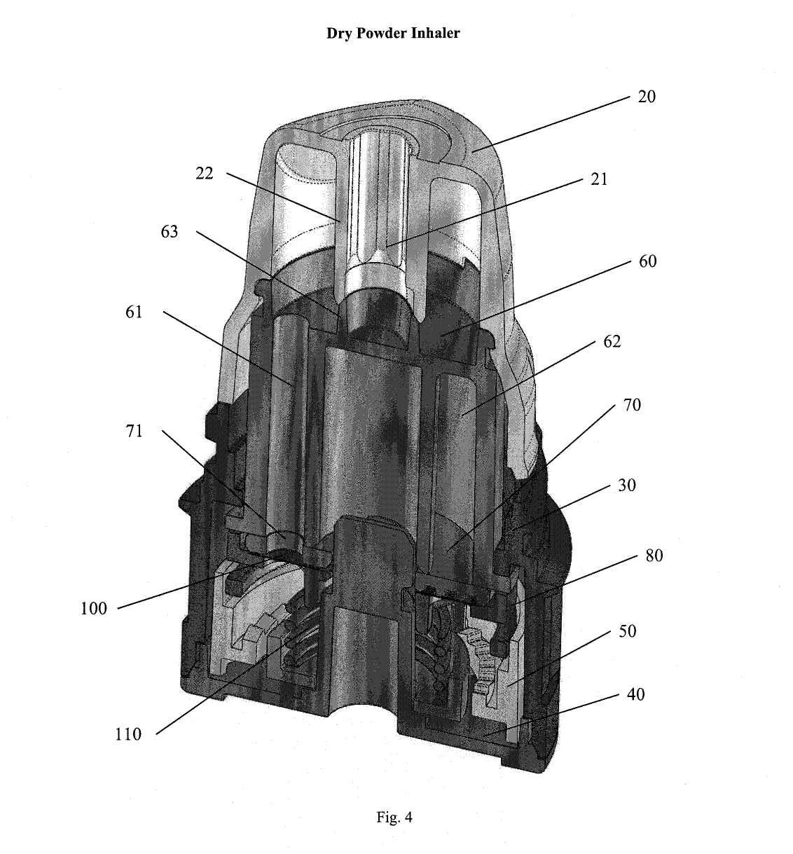

### DETAILED DESCRIPTION OF THE INVENTION

The Dry Powder Inhaler 1 includes the Overcap 10 that is removably attached to the Inhalation Device 2 by means of helix cams 31 (e.g., three sets) formed on the Adaptor 30. The helix cams 31 are engaged with the corresponding sets of the helix cam followers 12 formed on the Overcap 10. The Overcap 10 also includes pushers 11 (preferably three equally-spaced).

The Inhalation Device 2 is comprised of the Base 40, which is permanently attached to the Adaptor 30. The main features of the Base 40 are the post 41, the key 42, the equally spaced teeth 43 (preferably four), the disengagement cams 44 (preferably two), and the stopper 45. The Adaptor 30 also includes the dose counter window 34, the shoulder 33, and the locking slots 32 (preferably three).

The Counter Ring 50 is rotatably attached to the Base 40. The main features of the Counter Ring 50 are the driving teeth 41 (in the preferred embodiment there are 32 of such teeth—for a 30-day or one month supply, the one position reserved for locking of the device after the final dose is delivered), the disengagement teeth 52 (also 32 of them), the remaining dose counter numbers 53, and the stopper 54.

The Pawl 90 and the Support Plate 80 are rotatably positioned on the post 41 of the Base 40, and the keys 92 (preferably two) of the Pawl 90 are engaged with the corresponding key slots 83 of the Support Plate 80. The Pawl 90 also includes the driving arms 91 (preferably two), and the cam followers 93. The Support Plate 80 also includes the key slots 81 (preferably two), the spring arms 82 (preferably two), the air passage hole 85, and the key slot 86.

The Mesh 100 is permanently attached—preferably by means of ultrasonic welding—to the bottom ribs 74 of the Dose Plate 70. The main features of the Dose Plate also include the powder dose hole 71, and the key slot 73. The Dose Plate 70 with the Mesh 100 are positioned on the post 41 and the key 42 is engaged with the key slot 73 that prevents the rotation of the Dose Plate 70 relative to the Base 40. The Spring 110 is positioned in the pre-loaded compressed state on the post 41 between the Pawl 90 and the Support plate 80 and provides the axial force that sequentially engages the Support Plate 80, the Mesh 100, the Dose Plate 70, and the Powder Housing 40. The Powder Housing 40 is rotatably engaged with the shoulder 33 of the Adaptor 30 and rigidly connected to the Support Plate 80 by means of the two keys 65 that are engaged with corresponding key slots 81 of the Support Plate 80, and with the bottom surface of the Powder Housing 60 slidebly engaged with the top surface of the Dose Plate 70. The other main features of the Powder Housing 60 are the powder reservoir 62 for containing the powder medication (not shown), the inhalation conduit 61, the helix flow guide 63, and the air passage window 64. The Mouth Piece 20 is permanently attached to the Powder Housing 40, and the helix flow guide 63 of the Powder Housing 60 is vertically engaged with the corresponding helix flow guide 22 of the Mouth Piece 20. The other main features of the Mouth Piece include the inhalation hole 21, the air passage window 23, and the three driving arms 24.

The device operates generally in similar fashion to U.S. Pat. No. 6,240,918. In particular, the device operates as following:

In the initial position, the Overcap 10 is attached to the Inhalation Device 2, and the powder dose hole 71 of the Dose Plate 70 is located under the powder reservoir 62 of the Powder Housing 60, resulting in the powder dose hole 71 being filled with powder medication. The dimensions of the powder dose hole 71 define the volume of a dose to be administered. In this position, the driving arms 24 of the Mouth Piece 20 are biased inwardly by the pushers 11 of the Overcap, thus becoming disengaged from the locking slots 32 of the Adaptor 30. Also, the cam followers 93 of the Pawl 90 are positioned on the top portions of the disengagement cams 44 of the base 40, and the driving arms 91 of the Pawl 90 are positioned above the driving teeth 51 of the Counter ring 50. To use, the Overcap 10 is removed from the Inhalation Device 2 with a twisting motion. When the Overcap 10 is being removed from the Inhalation Device 2, the pushers 11 engage the driving arms 24, thus rotating the Mouth Piece 20 along with the other parts that are engaged with it and can rotate relative to the Base 40—the Powder Housing 40, the Support Plate 80, the Pawl 90, and the Spring 110. With sufficient rotation, the Overcap 10 is removed from the Inhalation Device 2. When the Overcap 10 is fully disengaged from the Device 2, the inhalation conduit 61 of the Powder Housing 60 is aligned with the powder dose hole 71 of the Dose Plate 70 and air passage hole 85 of the Support Plate 80. The dose may then be inhaled from the powder dose hole 71, through the inhalation conduit 61 and delivered through the Mouth Piece 20. When the Overcap is being rotated during the disengagement from the Inhalation device 2, dose counting is also achieved when the cam followers 93 of the Pawl 90 move along the profile of the disengagement cams 44 of the base 40, gradually lowering the driving arms 91 and engaging them with the driving teeth 51 of the Counter Ring 40 advancing it to the next position and decrementing by one the counter number 55 visible through the counter window 34. After use, the Overcap 10 is re-attached to the Inhalation Device 2 causing rotational movement and alignment of the components in the initial state. Every time the Overcap 10 is attached and removed the cycle is repeated until the stopper 54 of the Counter Ring 50 reaches the Stopper 45 of the Base 40.

Although there is some operational similarity to U.S. Pat. No. 6,240,918, differences do exist as can be seen.

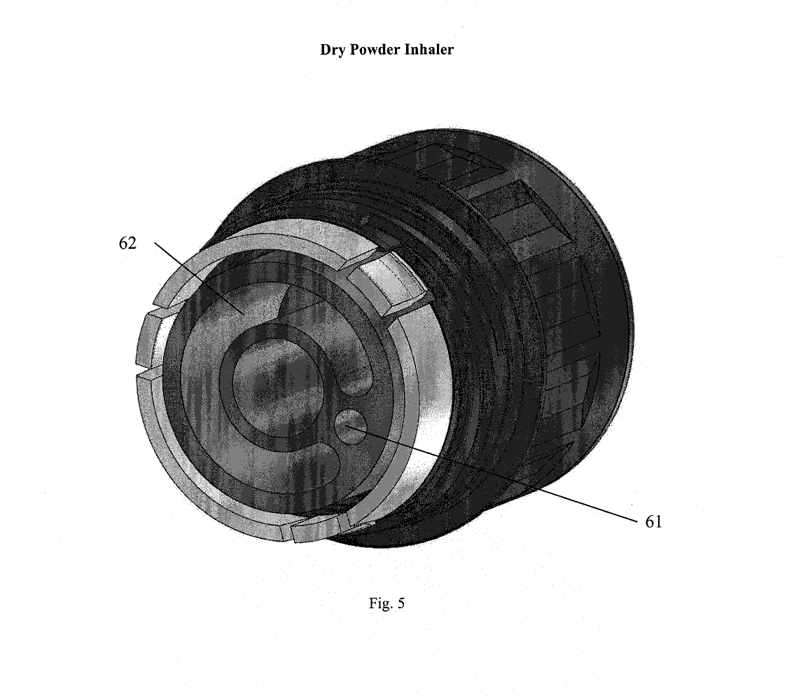

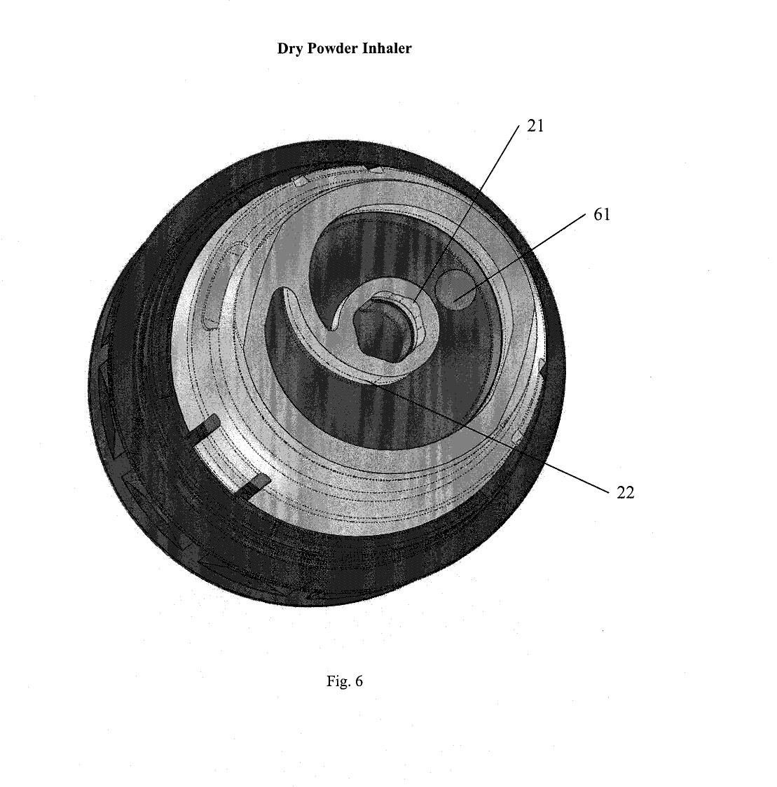

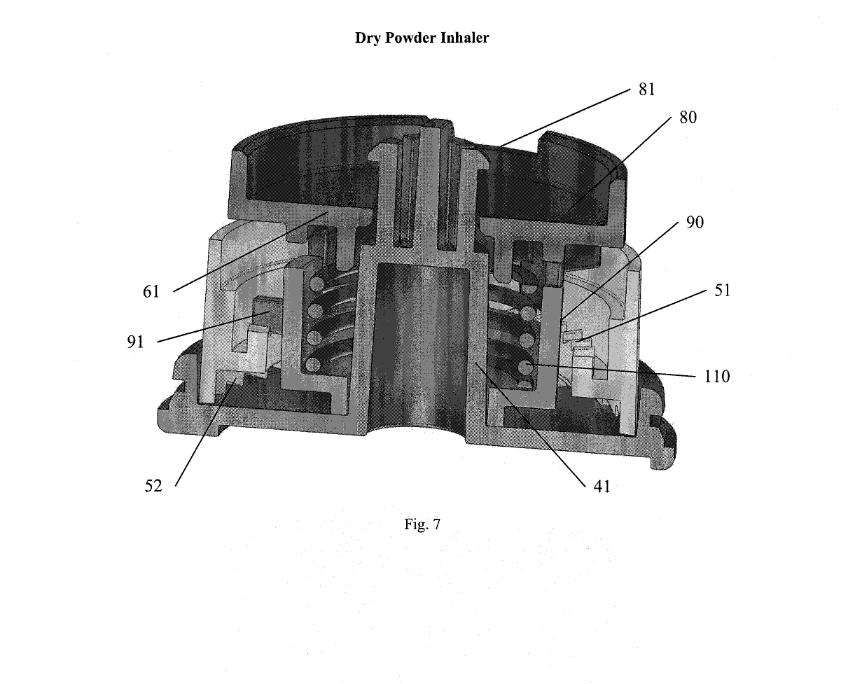

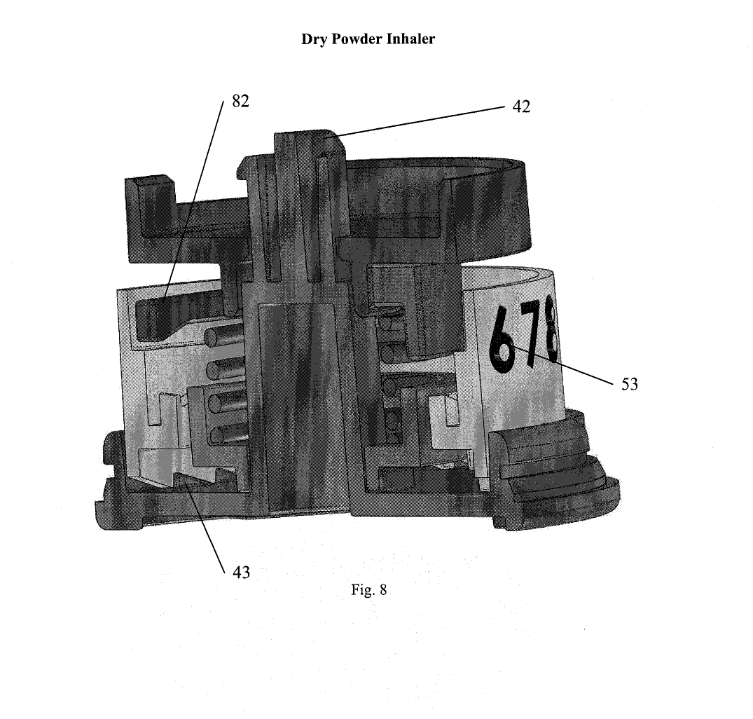

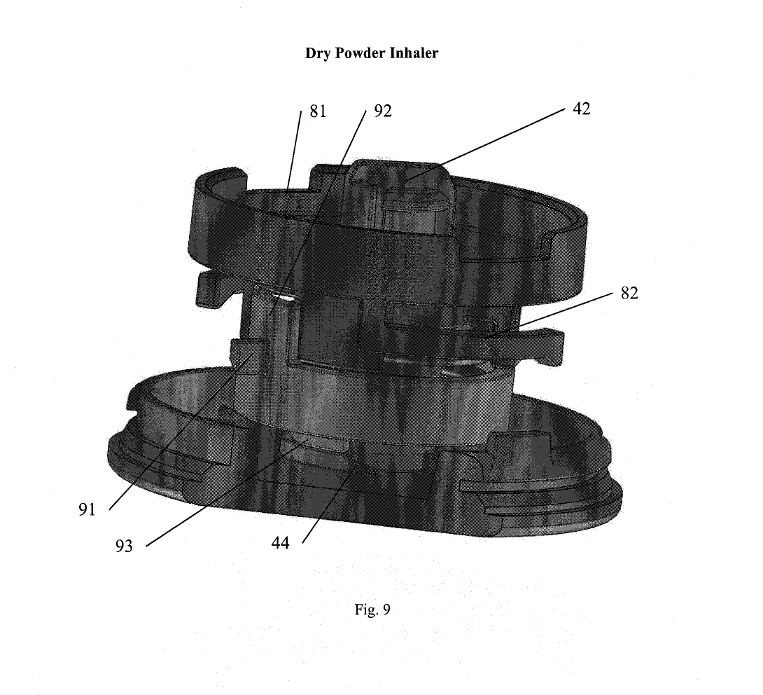

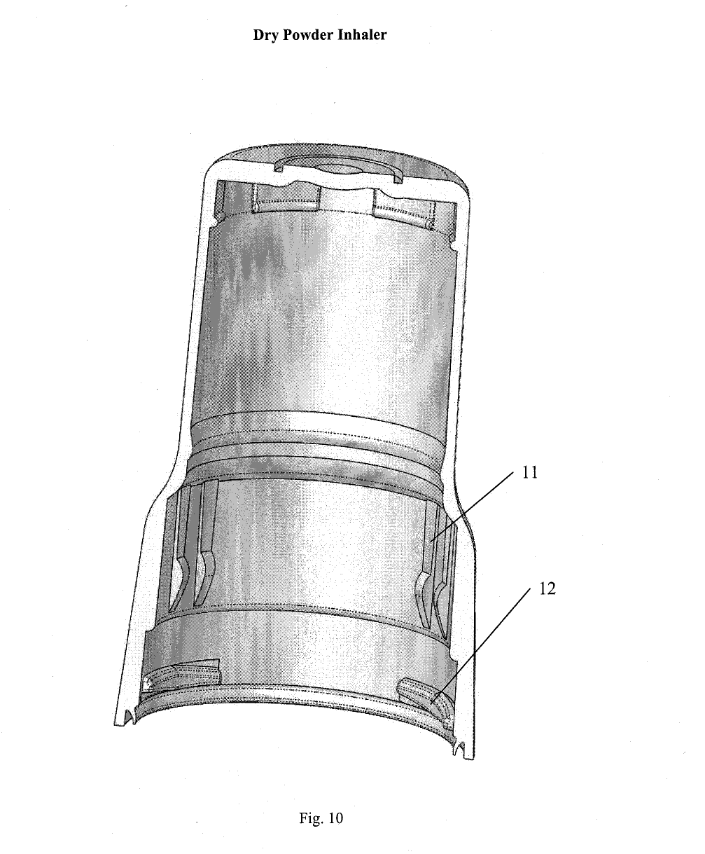

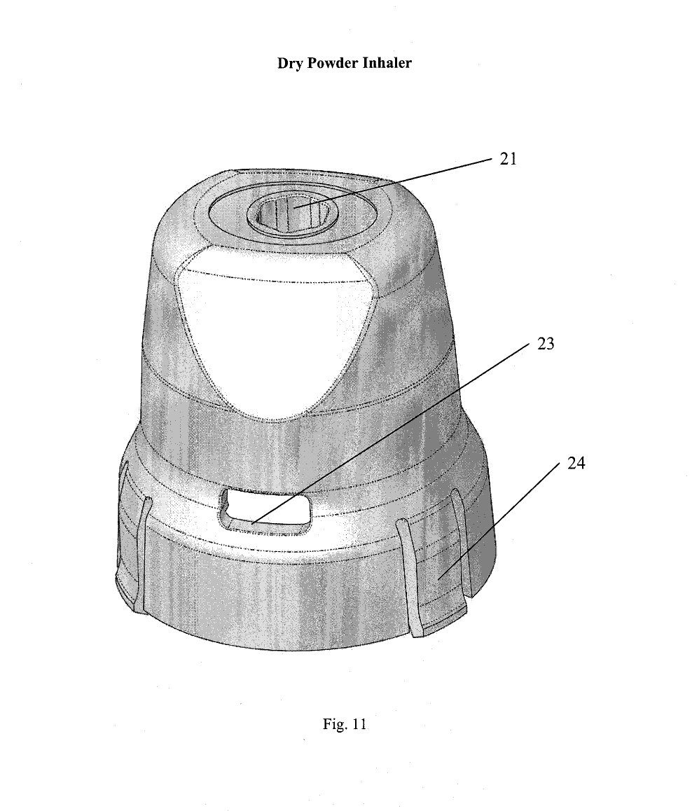

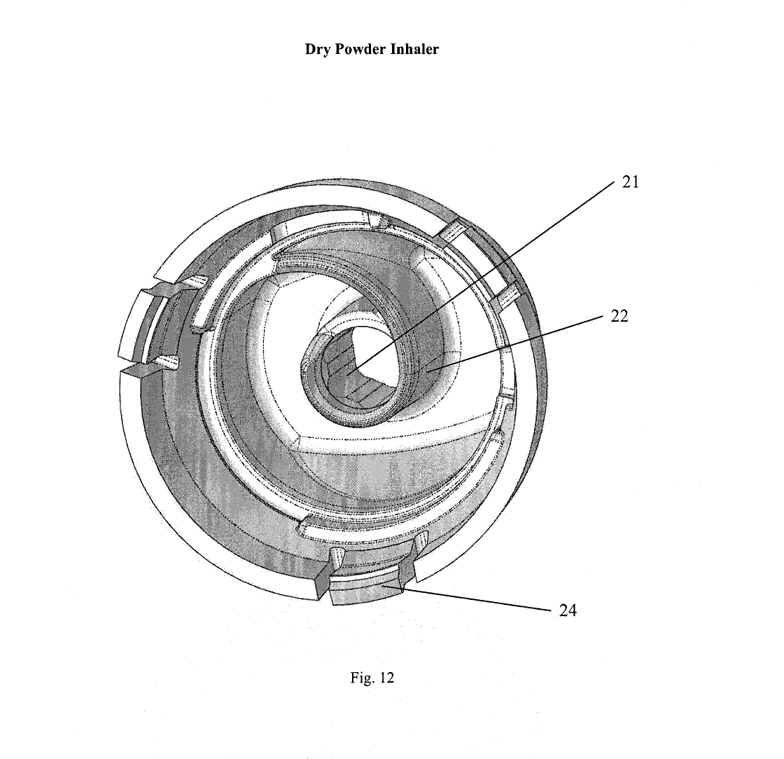

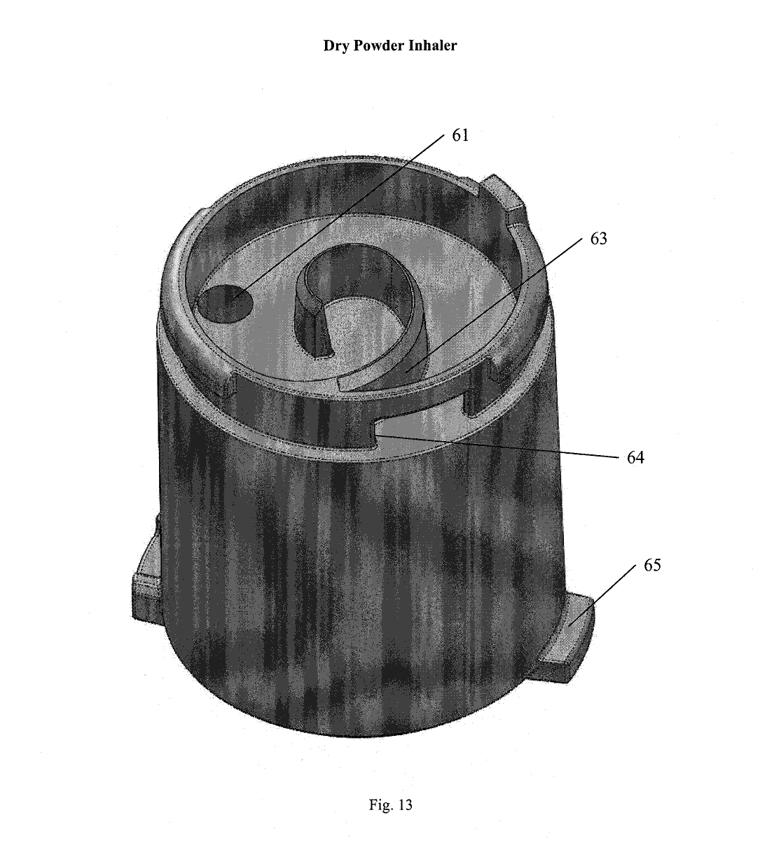

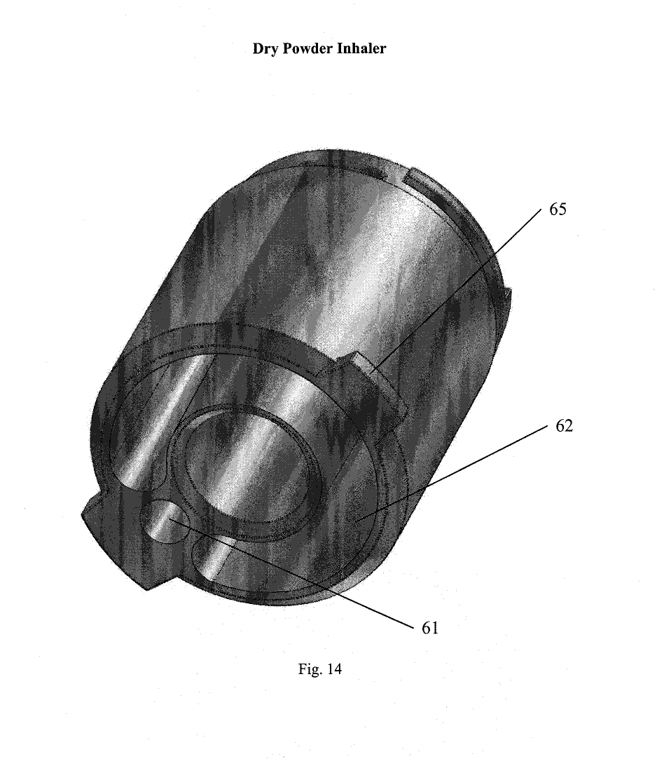

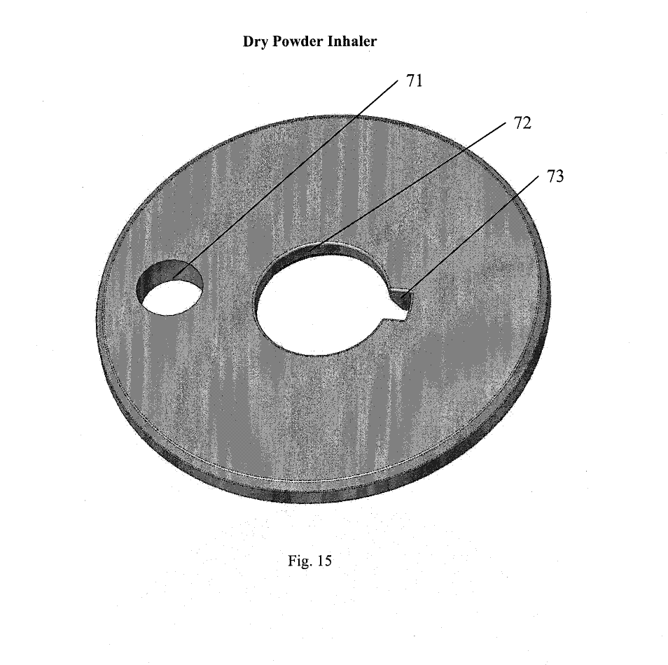

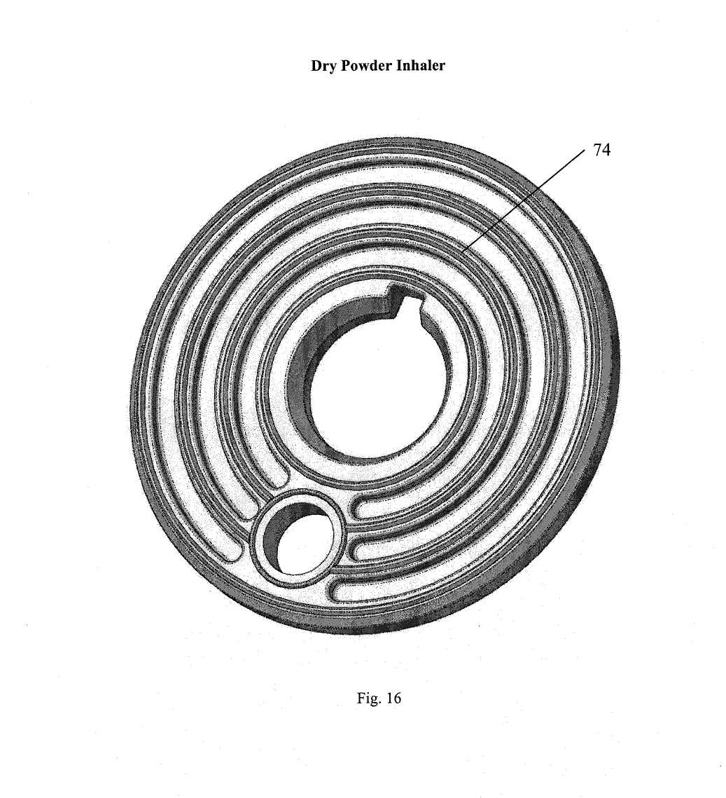

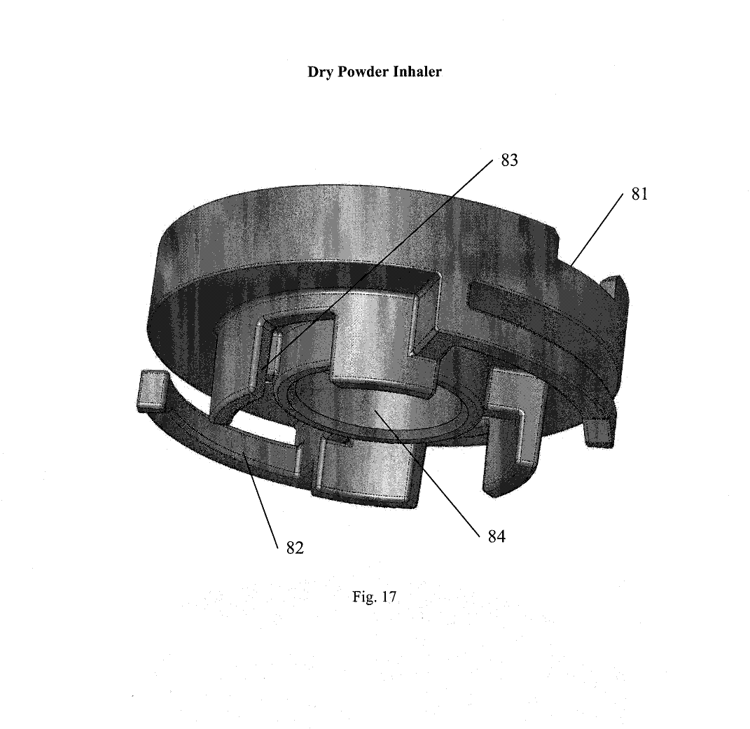

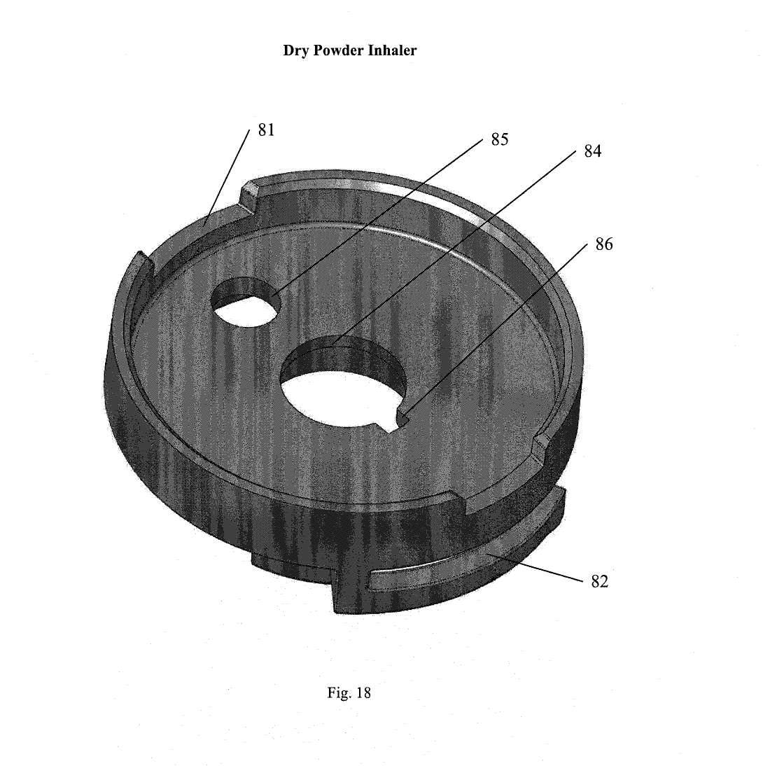

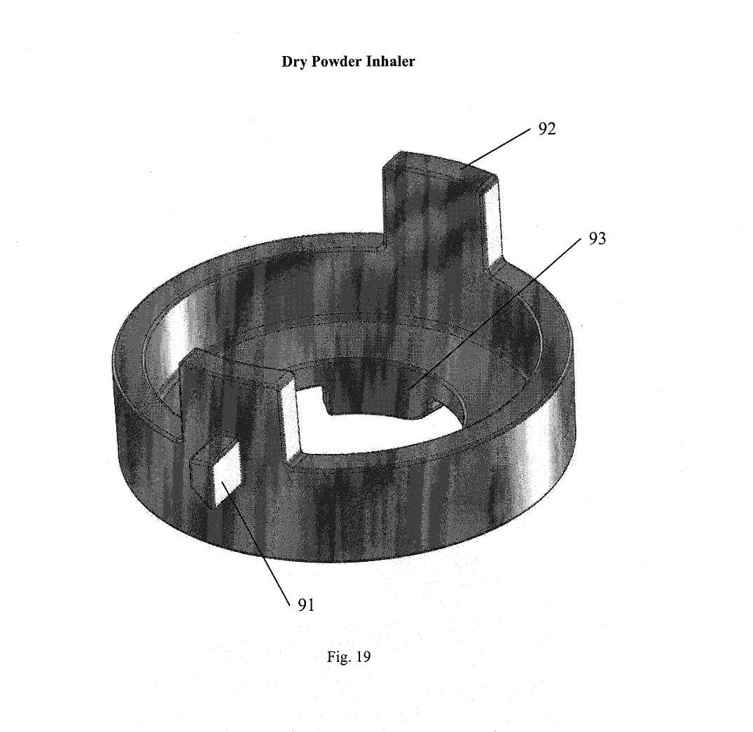

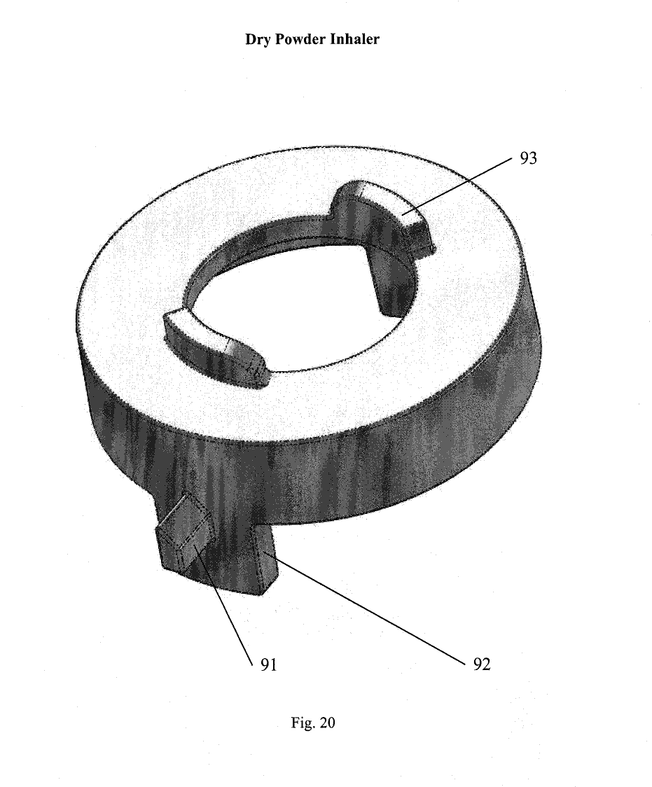

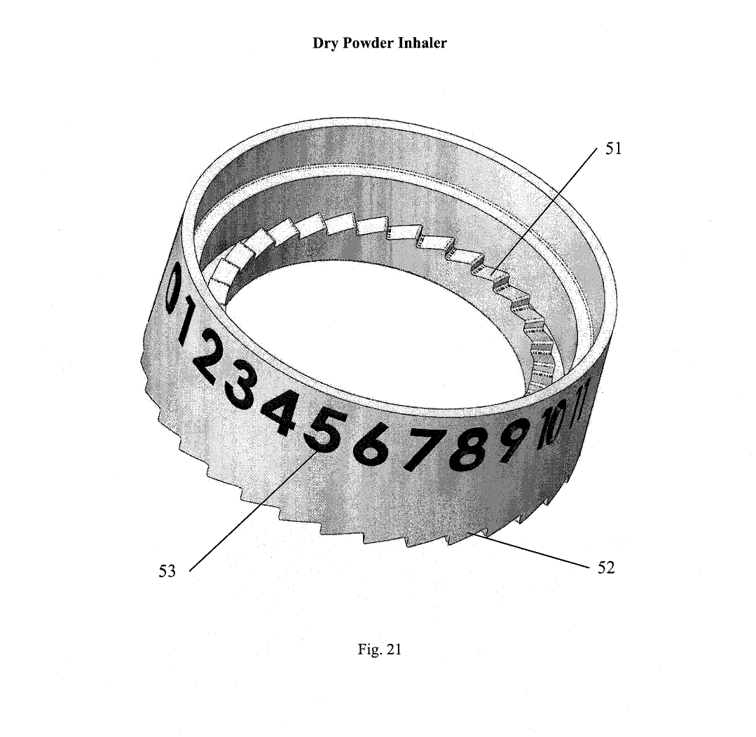

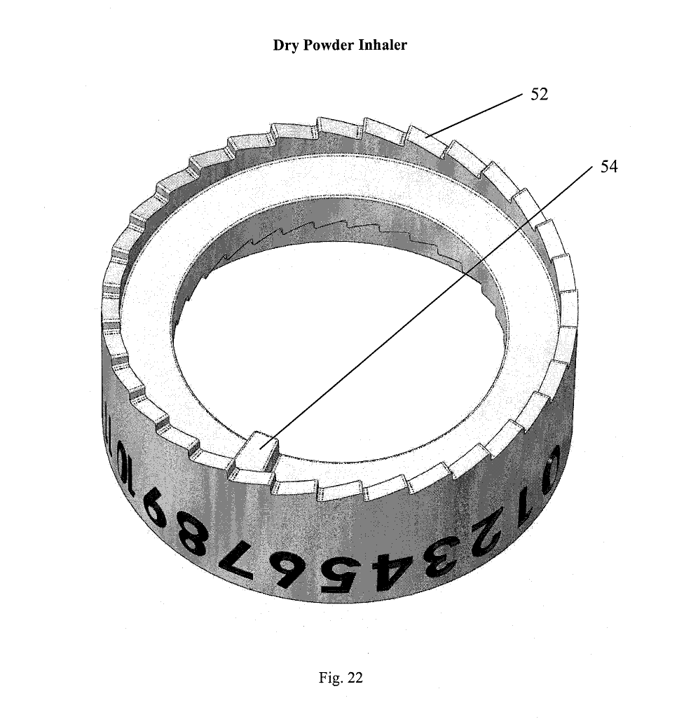

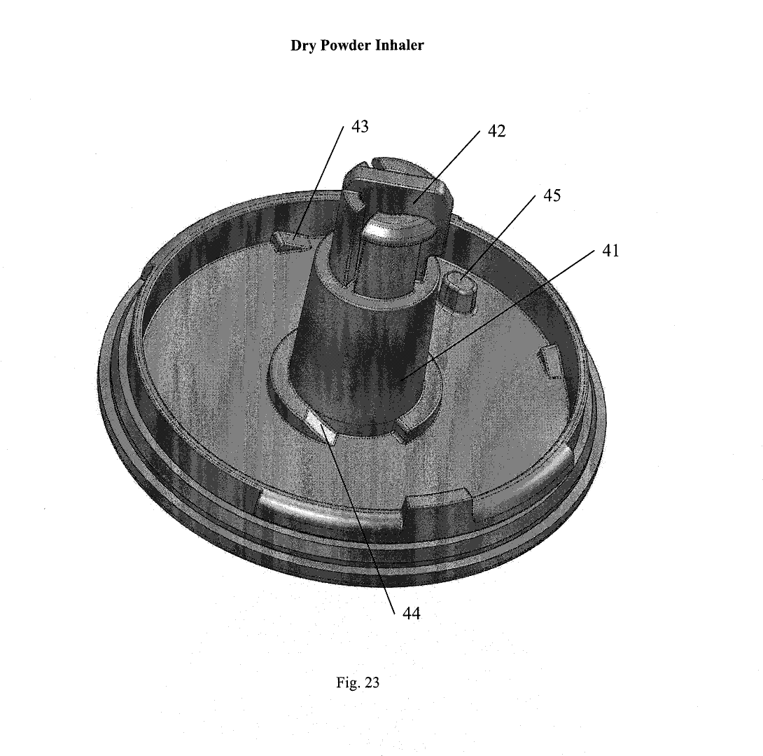

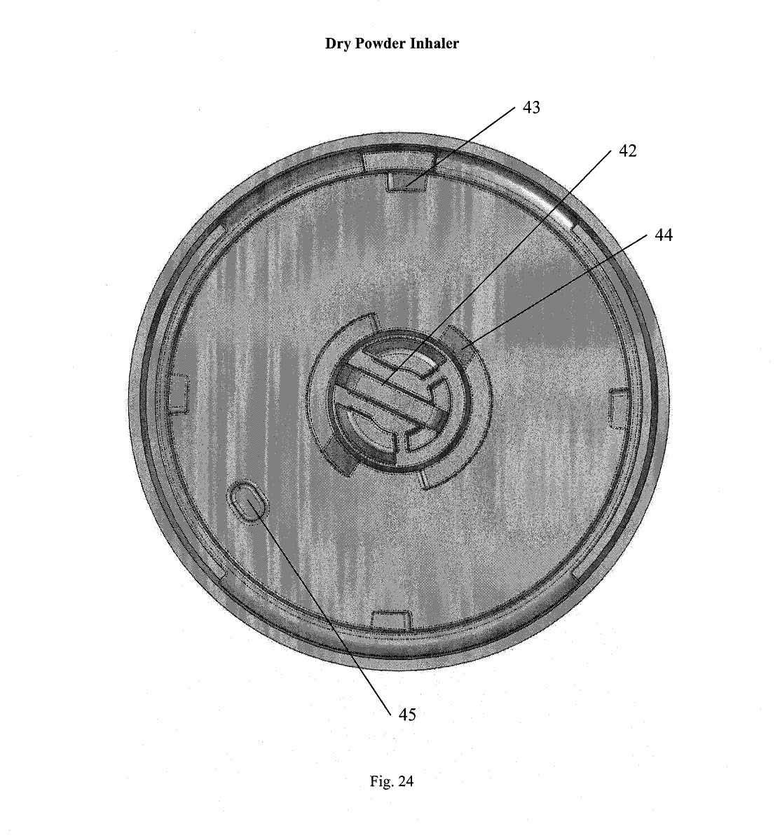

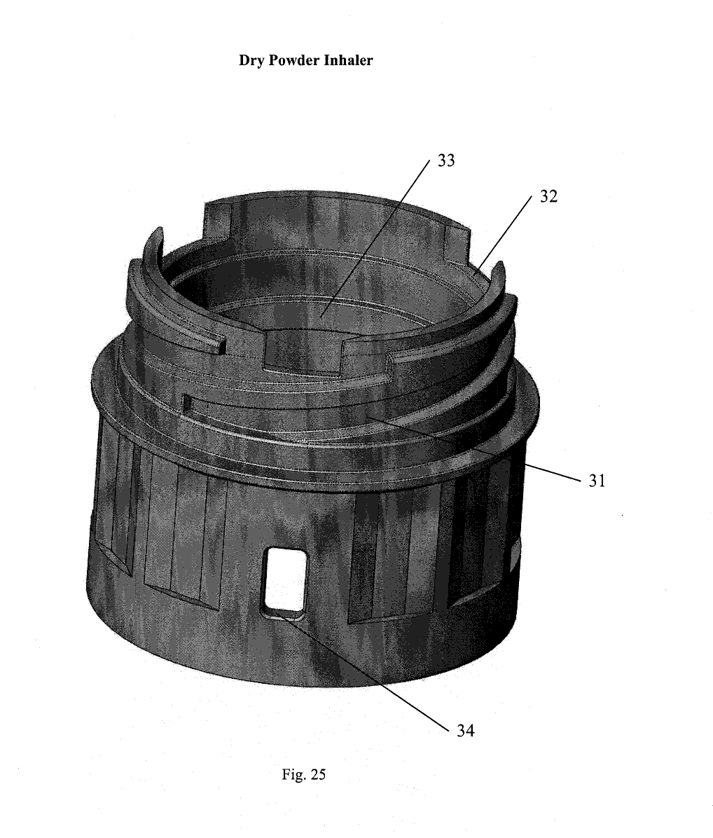

As will be appreciated by those skilled in the art, with reference to FIG. 5, the Reservoir 62 has a generally crescent-shaped profile which extends substantially (more than half) about a circumference of the Powder Housing 60. The Reservoir 62 terminates at end points between which is located the inhalation Conduit 61. With this arrangement, during dose preparation, the dose hole 71 will traverse below the Reservoir 62 along a substantial portion of the relative movement therebetween. The dose hole 71 will only separate from the Reservoir 62 upon coming into substantial alignment with the Inhalation conduit 61. The dose hole 71 may be configured to be in a state where it transiently is in simultaneous communication with the Reservoir 62 and the inhalation conduit 61 during relative movement between the Dose Plate 70 and the Reservoir 62. By allowing for such great exposure between the Reservoir 62 and the dose hole 71, a larger number of does and/or larger doses may be achieved. This is achievable by allowing a greater working length of the Reservoir 62 to be exposed to the dose hole 71 during relative movement between the Dose Plate 70 and the Reservoir 62; this allows for more medication to be made accessible to the dose hole 71.

What is claimed is:

1. A dry powder inhaler comprising:
a housing defining a reservoir for accommodating dry powder, said reservoir having a generally crescent-shaped profile which extends substantially about a circumference of said housing.

2. A dry powder inhaler as in claim 1, further comprising a dose plate adjacent to said reservoir, said dose plate defining a dose hole which is selectively communicatable with said reservoir with relative movement between said dose plate and said reservoir.

3. A dry powder inhaler as in claim 2, wherein said reservoir terminates at end points, and, wherein, an inhalation channel is located between said end points isolated from said reservoir.

4. A dry powder inhaler as in claim 3, wherein said dose hole is selectively positionable to be in alignment with said inhalation channel.

5. A dry powder inhaler as in claim 4, wherein said dose hole is configured so as to be in a transient state in simultaneous communication with said reservoir and said inhalation channel during relative movement between said dose plate and said reservoir.
